(19) United States
(12) Patent Application Publication
(10) Pub. No.: US 2008/0269753 A1
(43) Pub. Date: Oct. 30, 2008

(54) DYNAMIC CERVICAL PLATE

(75) Inventor: Andrew F. Cannestra, Jacksonville, FL (US)

Correspondence Address: WARNER NORCROSS & JUDD LLP, 900 FIFTH THIRD CENTER, 111 LYON STREET, N.W., GRAND RAPIDS, MI, 49503-2487, US

(73) Assignee: BLUE FURY CONSULTING, LLC, JACKSONVILLE, FL (US)

(21) Appl. No.: 12/108,627

(22) Filed: Apr. 24, 2008

Related U.S. Application Data
(60) Provisional application No. 60/914,144, filed on Apr. 26, 2007.

(51) Int. Cl. A61B 17/56; A61B 17/80

(52) U.S. Cl. 606/70; 606/280

(57) ABSTRACT

A cervical plate assembly having a body, with at least two wings, and a tensioning mechanism for applying a force to the wings. The tensioning mechanism includes a shape memory band and an adjuster for shortening or lengthening the band.

### CROSS REFERENCE TO RELATED APPLICATIONS

This application claims the benefit of priority under 35 U.S.C. 119(e) to U.S. Patent Application 60/914,144, filed Apr. 26, 2007, which is incorporated herein by reference in its entirety.

### BACKGROUND OF THE INVENTION

The present invention relates to anterior cervical plates and more particularly to dynamic cervical plates.

Spinal fusion surgery has become a common procedure for the treatment of degenerative disease of the spine. In such surgery, spinal hardware is installed to stabilize the movement across the affected joint, thereby facilitating fusion between the vertebrae. Historical spinal hardware includes plates, screws, and rods.

Anterior cervical fusion necessitates the use of an interbody spacer or cage between the affected levels to replace the removed disc. Anterior cervical plates are then applied across the levels to increase stabilization and promote fusion. Cervical plates of the present type are generally elongated so as to span the distance between two, three, four or more vertebrae, as required in a given situation. Cervical plates are almost exclusively static, in that they have fixed dimensions.

It is desirable in certain situations to allow shifting or slight movement between the plate-mounted vertebrae. This movement is desirable because during the first six weeks post implant, the interbody spacers may subside within (also known as piston into) the adjacent vertebrae. This subsidence results in the shortening of the distance over which the plate must span. Unfortunately, a static plate can not shorten, and therefore fails to load-share during subsidence. In extreme cases, plate or screw failure can occur.

To mitigate subsidence and plate failure, dynamic plates have been developed which allow motion during subsidence. As dynamic plates shorten during subsidence of the spacers, the plates continue to load-share. Thus dynamic plates restrict motion, but do not eliminate or stabilize against motion. Dynamic plates may also allow the unloading of the graft when the patient is reclined (i.e. no weight or force placed across the dynamic plate). Use of a dynamic plate can therefore lead to micro-motion across the fusion plane, failure to fuse and hardware failure.

Generally, in order to allow movement, the dynamic plates include either sliding plates or plates that allow the screws to pivot or slide within the plate. Some dynamic plate designs have mechanisms to ratchet or otherwise control the shortening action of the plate. Unfortunately, each ratchet location creates an increase in the force asserted.

### SUMMARY OF THE INVENTION

The present invention provides a dynamic cervical plate including a tensioning mechanism to provide both (a) a resistive force across the fusion level and (b) a compressive force to stimulate bone growth. The present invention thereby provides both a compressive force and a resistive force across the fixated level.

In the current embodiment, the plate includes a sliding mechanism and an adjustable tension wire or band. The tension band has structural integrity so that is also provides a resistive force to compression. By means of the adjustable tension band, the compressive and resistive forces can be adjusted in-situ during the operative procedure.

The present invention has at least three advantages over prior art plates. First, it allows for application of compressive force across a fusion construct. Second, this applied force is adjustable at the time of insertion. Third, as subsidence occurs (in the approximately six weeks following the procedure), the tension band provides structural support by resisting subsidence.

These and other objects, advantages, and features of the invention will be more fully understood and appreciated by reference to the description of the current embodiments and the drawings.

### BRIEF DESCRIPTION OF THE DRAWINGS

FIG. 1 is a top plan view of the dynamic cervical plate of the present invention;

FIG. 2 is a top plan view of the tensioning mechanism;

FIG. 3 is a top plan view of the tensioning mechanism applied to a slot type plate.

FIG. 4 is a top plan view of an alternative cervical plate in which the tensioning mechanism is contained entirely within the plate body;

FIG. 5 is a top plan view of an implanted cervical plate with the vertical arrows indicating the direction of the compressive force;

FIG. 6 is a top plan view of the tensioning mechanism during the tensioning process with the arrows indicating the increased tensioning of the device;

FIG. 7 is a top plan view of an implanted cervical plate in its final configuration after adjusting the tensioning mechanism; and

FIG. 8 is a top plan view of the tensioning mechanism in its final configuration after adjusting the tensioning mechanism.

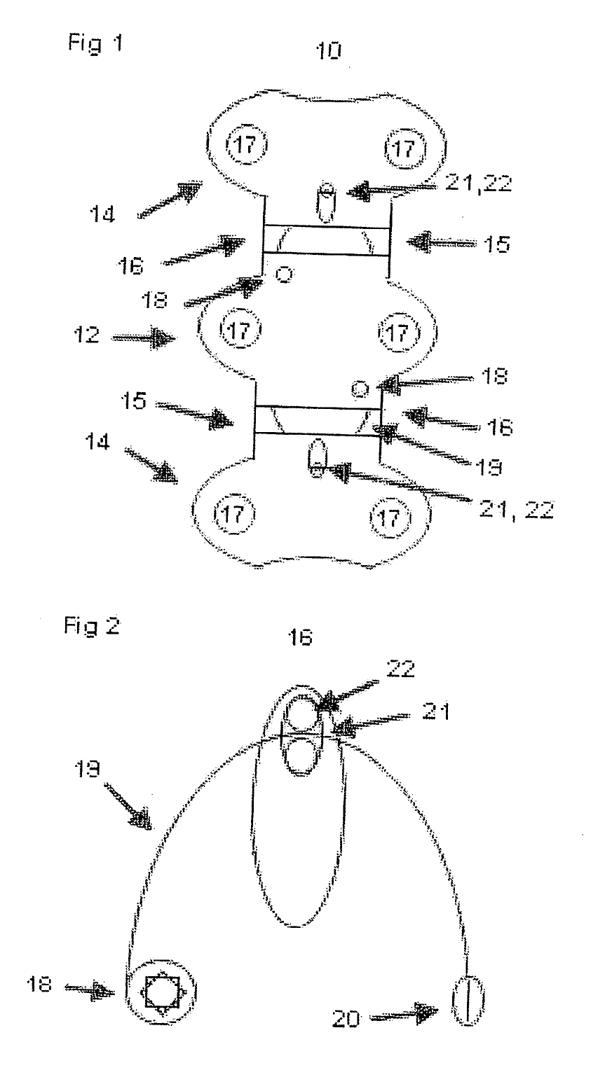

### DESCRIPTION OF THE CURRENT EMBODIMENTS

A dynamic cervical plate constructed in accordance with a current embodiment of the invention is illustrated in the drawings and generally designated 10.

The plate 10 includes a plate body 12 and dynamic, ends or wings 14 that can slide relative to the plate body to shorten the overall length of the plate 10. The plate body 12 and the plate wings 14 are each preferably fabricated of a single piece, but could be fabricated of multiple pieces. Suitable materials for the plate body 12 and the wings 14 are and will be known to those skilled in the art, examples of which include but are not limited to, an alloy of metals, titanium, a titanium alloy, PEEK, or other suitable biocompatible material.

The body 12 can also include a coating on the exterior surface that promotes bone growth. The coating can include osteoinductive agents (including growth factors, cell therapy, gene therapy, and patient derived factors) and other drug therapies. The osteoinductive agents can be added to initiate and accelerate bone formation. The drug therapies can range from antibiotics to reduce the risk of infection to chemotherapy to treat cancer.

Optionally, the plate can be used with a flowable bone filler material. As used herein, bone filler is defined as any substance used to stabilize the bone and includes, but is not limited to bone cement (polymethyl methacrylate (PMMA), or (PMA)), other composite material, human bone graft (allograft or autograft), synthetic and xenograft derived bone substitutes (calcium phosphate, hydroxylapatite, and/or other ceramic based bone substitutes), collagen, or combinations of these materials made of titanium, or other biocompatible materials such as stainless steel, graphite, carbon fiber materials, PEEK, nitinol, or various plastics and composites of the foregoing.

Various materials may be used for the grafts installed during use. Examples of such materials include, but are not limited to, titanium, ceramic and nylon inserts. Further, the materials can include allografts taken from long bones such as the femur, humerus, tibia and fibula. Allografts are removed from a donor and processed using known techniques to preserve the allograft until implantation. Allografts have mechanical properties which are similar to the mechanical properties of vertebrae even after processing. The benefit of such property matching is that it prevents stress shielding that occurs with metallic implants. Allografts, unlike magnetic metals, are also compatible with magnetic resonance imaging (MRI) procedures, allowing more accurate ascertainment of fusion. Furthermore, allografts are naturally osteogenic providing excellent long term fusion with the patient's own bone.

The plate body 12 and the dynamic wings 14 include holes 17 for the placement of bone screws into the underlying vertebral bones. The holes 17 are configured to utilize various types of bone screws such as fixed angle screws, emergency screws, and variable angle screws, examples of which are known to those of skill in the art. Moreover, the holes 17 allow variable bone screw angulation while fixing or mounting the plate to the vertebrae.

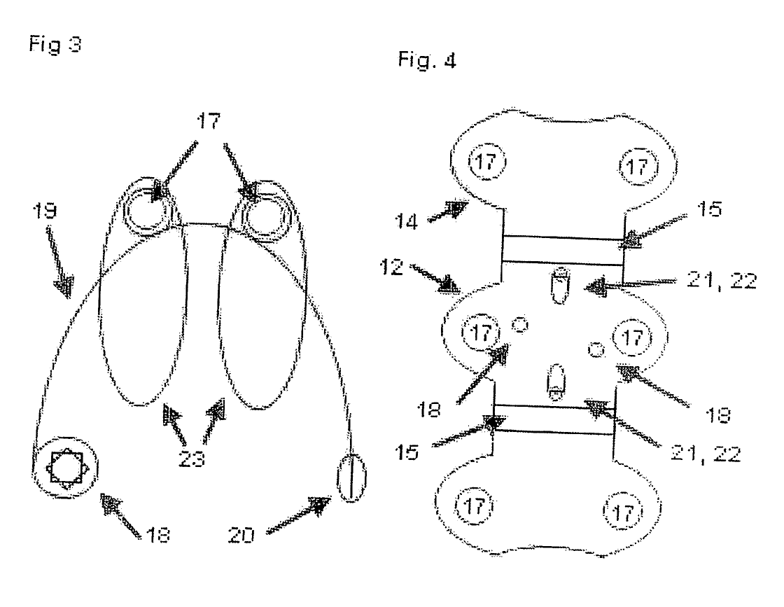

As seen in FIG. 3, die plate body 12 includes both an anchor point 20 for the tension band 19 (to be described), and also a tensioning adjustment cam 18 for the band 19. The band 19 passes through a slot 21 on the plate sliding pin 22. The band 19 will slide freely through the sliding pin 22, thereby allowing the band to be shortened or lengthened by means of the adjustment cam 18. The adjustment cam or device 18 is within the plate body 12 and can shorten the tension band 19, thereby providing increased force between the plate body 12 and plate wing 14.

Across the wings 14, and spanning the gaps 15 over which the plate can shorten, is the tensioning mechanism 16 including a tension wire or band 19. The tensioning assembly 16 is illustrated in FIGS. 2-3, 6, and 8. The band is anchored in two locations: 1) the plate body anchor point 20 and 2) the adjustment cam or device 18. The band 19 passes freely through the slot 21 in the dynamic sliding pin 22. The adjustment cam 18 preferably includes a ratcheting or other movement control device (not shown) to prevent inadvertent movement and undesired tension band adjustment.

The tension band 19 is fabricated a material that is both flexible and provides support. The material can be Nitinol or other shape memory material to provide a flexible tension band with structural “memory” (shape memory materials) for support. Additionally, the wire can be formed from stainless steel, cobalt-chrome alloy, titanium, titanium alloy. It is further contemplated that the metallic wire can be interwoven with non-resorbable polymers such as nylon fibers, carbon fibers and polyethylene fibers, among others, to form a metal-polymer composite weave, single filament and multi-filament expanded polytetrafluoroethylene (PTFE), a single or multi-filament polyethylene terephthalate (PET), lightly or tightly braided polyester filaments, bioabsorbable materials such as poly-L-lactide (PLLA), polyglycolic acid (PGA) suture, PLA or polylactide suture, copolymers of PGA/PLA such as poly(lactide-co-glycolide), polydioxanone (PDS) suture, polycaprolactone and polyhydroxybutyrate (PHB). The wire can also be modified in a number of ways, including electrochemical surface modifications, coating applications and thermal treatments. The material can be anodized, thermally hardened, interwoven with collagen material, include collagen molecules immobilized to its surface, coated/impregnated with an elastomer, adhesive or a therapeutic agent, or include alternating strands of metal wires and demineralized bone matrix or collagen. The wire can also be formed of a biopolymer capable of switching between at least two states, wherein the polymer can be forced into either a hard/stiff state or a pliable state. The biopolymer can therefore be positioned during the softened state and then once the desired position is obtained, change the configuration to the stiff state. This allows the band to be easily positioned within the plate.

### Installation/Use

Suitable procedures for inserting the dynamic anterior plate 10 across a disc space are known to those skilled in the art and will not be illustrated here. Briefly, the plate 10 is secured into place using bone screws (not shown) inserted through the screw holes 17.

Once in position, the adjustment cam 18 is rotated to provide the desired balance between tension or compressive force and support or resistive force. The adjustment cam 18 may be tightened or loosened to either lengthen or shorten the tension band 19, respectively. Shortening the band 19 will contract the distance between the plate body 12 and the wings 14. The tension band 19 will coil around the adjustment cam 18.

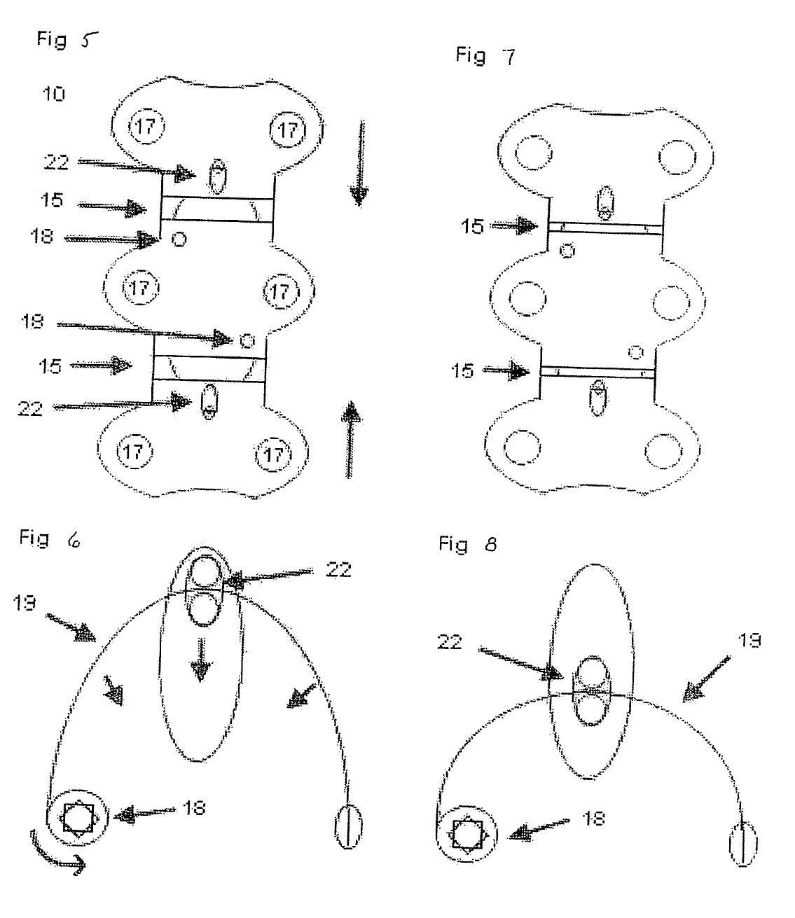

The adjustment of the tensioning band 19 is illustrated in FIGS. 5-8. Once the plate 10 is in place, a tool (not shown) is inserted into or about the adjustment cam 18. The adjustment cam 18 is then turned to adjust the amount of tension provided by the tension band 19 (FIG. 6). The tool could include an integral torque indicating device (e.g. a torque wrench type tool). The adjustment causes the tension band 19 to shorten and produce a compressive force across the dynamic portions of the plate 15 as indicated by the arrows in FIGS. 5-6 through the plate sliding pin 22. The excess portion of the tension band 19 is coiled around the adjustment cam 18 (not shown). When the appropriate tension is produced, removal of the tool from the adjustment cam 18 will lock the adjustment cam 18 in place to prevent inadvertent loosening. This process results in a shortened effective length of the tension band 19 and a compressed dynamic plate 15 (FIGS. 7-8).

The present invention provides a tensioning mechanism 16 to be applied to a dynamic anterior cervical plate 10. The mechanism contains adjustable tension and support characteristics that can be readily and securely installed on the spine to enhance fusion. The device creates a minimum of trauma and produces excellent fusion results.

The present invention is applicable to cervical plates of a number of configurations. Cervical plates are generally referred to by the number of levels that they overlie, wherein the word “level” refers to the number of intervening intervertebral spaces that are spanned. Thus, for example, a three level cervical plate would span the four vertebrae beyond and between the three intervertebral spaces. The current embodiment is for a two level dynamic plate. However, the present invention is adaptable for use as a 1-7 level cervical plate.

### Alternative Embodiments

The tensioning mechanism 16 may also be applied to slot type plates (FIG. 3) or contained within the plate body 12 so as not to span the dynamic space 15 (FIG. 4). In the FIG. 3 embodiment, the tensioning band is affixed to the plate body 12 adjacent the holes 17 through which the bone screws (not shown) are installed. The bone screws subsequently travel in the slots 23 within the plate. The bone screws can rest upon the tensioning mechanism 16. Alternatively, the tensioning mechanism 16 can attach to a ring (not shown), collar, or other device through which the bone screws can pass. In other embodiment, the can be welded or melded to the tensioning mechanism 16 upon insertion.

In the FIG. 4 embodiment, the tensioning band 19 is contained entirely within the plate body 12.

The tensioning mechanism 16 may also be applied to existing cervical plates. Thus, the plate does not have to been specifically designed or manufactured to include the tensioning mechanism 16. In such a “retro-fit” situation, the tensioning mechanism 16 is affixed to the existing plate using a bone cement or other similar adhesive. Alternatively, if the tensioning mechanism 16 is a metal, the metal can be sintered to the surface of the plate. When the tensioning mechanism 16 is formed of a shape memory material, the adjustments can be made by altering the amount of heat or current applied to the tensioning mechanism 16 until the band is in the proper configuration. The shape memory material shape can be adjusted based on the amount of heat or current applied. Alternatively, the tensioning mechanism 16 can be adjusted by twisting the material of the tensioning mechanism 16 on itself. The tensioning mechanism 16 can be shaped as a circle or C-shaped and placed within the dynamic space portion of the plate, wherein either end of the C can rest on the body 12 and wings 14 respectively. The ends of the C can be welded, glued, adhesed, or otherwise affixed in place either prior to or after insertion. This enables the tensioning band 16 to be added to existing plates.

The above descriptions are those of current embodiments of the invention. Various changes and alterations can be made without departing from the spirit and broader aspects of the invention.

The embodiments of the invention in which an exclusive property or privilege is claimed are defined as follows:

1. A cervical plate assembly comprising
a cervical plate having a body and a wing; and
a tensioning means for urging said wing to a position relative to said body.

2. The cervical plate of claim 1, wherein said tension band is a strand of material.

3. The cervical plate of claim 2, wherein said tension band is a braid formed of at least three of said stands of material.

4. The cervical plate of claim 2, wherein said strand of material is formed of a material selected from the group consisting essentially of a shape memory materials, a bioabsorbable material, a metal and biocompatible materials.

5. The cervical plate of claim 1, wherein said cervical plate includes anchoring means for anchoring said tension band in place.

6. The cervical plate of claim 5, wherein said tension band is adjustable.

7. The cervical plate of claim 6, wherein said cervical plate includes adjustment means for adjusting said tension band.

8. The cervical plate of claim 7, wherein said adjustment means is a cam.

9. The cervical plate of claim 1, wherein said plate includes sliding means interconnecting said wing and said body.

10. A cervical plate comprising;
a first plate portion adapted to be secured to a first vertebrae;
a second plate portion adapted to be secured to a second vertebrae; and
force generating means for generating a force between said first and second plate portions when attached toe the first and second vertebrae respectively.

11. The cervical place of claim 10, wherein the force is a compressive force.

12. A method of implanting a cervical plate by:
preparing a disc space for a cervical plate;
inserting the cervical plate having a tensioning mechanism into the prepared disc space; and
tensioning the tensioning mechanism to provide an appropriate amount of resistive support to the cervical plate while within the prepared disc space.
